(19) United States
(12) Patent Application Publication
(10) Pub. No.: US 2022/0036986 A1
(43) Pub. Date: Feb. 3, 2022

(54) Patient Specific, Condition Specific, and Treatment Specific Dental Treatment Plan

(71) Applicant: Gray Gray Petty, Salt Lake City, UT (US)

(72) Inventors: Robert Thorup, South Jordan, UT (US); David Mecham, Alpine, UT (US); Michael Geary, Provo, UT (US)

(73) Assignee: My Practice My Business, LLC, Orem, UT (US)

(21) Appl. No.: 15/808,999

(22) Filed: Nov. 10, 2017

(51) Int. Cl. G16H 20/00; G16H 10/60

(52) U.S. Cl. CPC G16H 20/00; A61C 8/00; G16H 10/60

(57) ABSTRACT

A dental treatment plan generator, which includes different options for patients to choose the material they would like used in their procedures. The treatment plan generator allows patient to see the details of the proposed procedure, along with detailed descriptions and diagrams of; the teeth involved, the history of the procedure, why the procedure is recommended, how the procedure will be carried out, the materials recommended, and cost comparisons between materials. Patients therefore gain confidence in and control over their procedures.

### TECHNICAL FIELD

Dental treatments. Specifically, treatment procedures and options regarding materials.

### BACKGROUND

The invention relates generally to dental health. More specifically the invention relates to helping dentists and their staff educate a patient about a dental procedure. Dental procedures can be particularly difficult to describe. Oral descriptions often leave patients confused. Detailed printed descriptions with accompanying photos and diagrams enable dentist to inform patients in a more understandable way, the procedures necessary.

The current state of dentistry relies on dentists and their assistants, describing to their patients the issues that have been found and the solutions to those problems orally. There are occasionally plastic or plaster models of mouths with teeth to aid in the description. These techniques for describing complex oral issues often leave the patient more confused than enlightened

Dentistry has a particular jargon, one that is extremely descriptive and useful to those who understand the terminology and the physiologic structures that correspond to the dental terms. For those unfamiliar with the technical dentistry terms the descriptions can sound like a five-minute dissertation on alien anatomy.

Another shortcoming of the present state of the field is that these descriptions do not encourage patients to study the matter for themselves. It is difficult to remember a conversation at the end of the day, it is even more difficult to remember and be able to describe, even to oneself, a technically intense description filled with unfamiliar words. Even with the aid of plastic or plaster casts of the mouth, which are not available for patients to take with them, it can be difficult to correctly recreate the description given by the dentist, this gives the patient little opportunity to contemplate the proposed procedure in the comfort of their own abode. While wonderfully effective for dental procedures, dental offices are not the most effective place for personal reflection and decision making.

### SUMMARY OF THE INVENTION

The present invention solves one or more of the foregoing problems in the field of dentistry, relating to helping patients understand the procedures being described to them, and allowing them the ability to make a decision in a location away from the dental office.

For example, a method in accordance with the implementation of the present invention involves receiving input regarding the condition of the patient's teeth. Generally, the input includes several aspects including but not limited to, location in the mouth, type of issue, prescribed remedy. The software can be configured so as to give several options for materials used in treatment.

We use a database system to retain detailed information as to various dental conditions, procedures, methods for treating those conditions, different possibilities as to the materials used in the procedures and a program to select the information corresponding to a particular patient and to modify it as a dentist may require for that patient.

In essence, the database includes various templates of information as they relate to providing the patient's required knowledge and understanding of (a) Their Condition (Why dental treatment is needed); and (b) A brief overview of the procedure (How the treatment will be done) (c) The recommendation of the doctor (The material(s) and procedure(s) recommended for their condition) (d) Information specific to their treatment (tooth number, cost, etc.) These templates, which may be called “default templates,” result from the user providing information to a database or computer relating to the patient, procedure, and the like (i.e., answering a series of questions put by a computer relating to these matters). They can then be accepted as is or be customized as necessary for any unique problems of the patient. They are then formatted and printed. In the event that the user does not customize the templates, the selected default templates are printed as is.

The result is a personalized patient document which can be given to the patient, telling what condition he is suffering from, what procedure will alleviate that condition, the recommendations of the dentist, along with options for the patient to choose materials (specifically related to cost, durability and appearance, what post-operative procedures to follow, and explaining details of recovery patterns. The system has the advantage that use of the database and of a computer created check list assures that all necessary information will be provided, that any individual problems will be resolved in advance, and that the patient will have all necessary information in writing. Because of its completeness, it increases the knowledge of the patient and therefore increases the likelihood they will accept treatment.

### BRIEF DESCRIPTION OF THE DRAWINGS

A more particular description of the dental treatment software summarized above is made below by reference to specific embodiments. Several embodiments are depicted in drawings included with this application, in which:

FIG. 1 depicts a treatment plan for tooth fillings

FIG. 2 depicts a treatment plan for bone augmentation

FIG. 3 depicts a treatment plan for tooth replacement

FIG. 4 depicts a flow chart for generation of the patient document

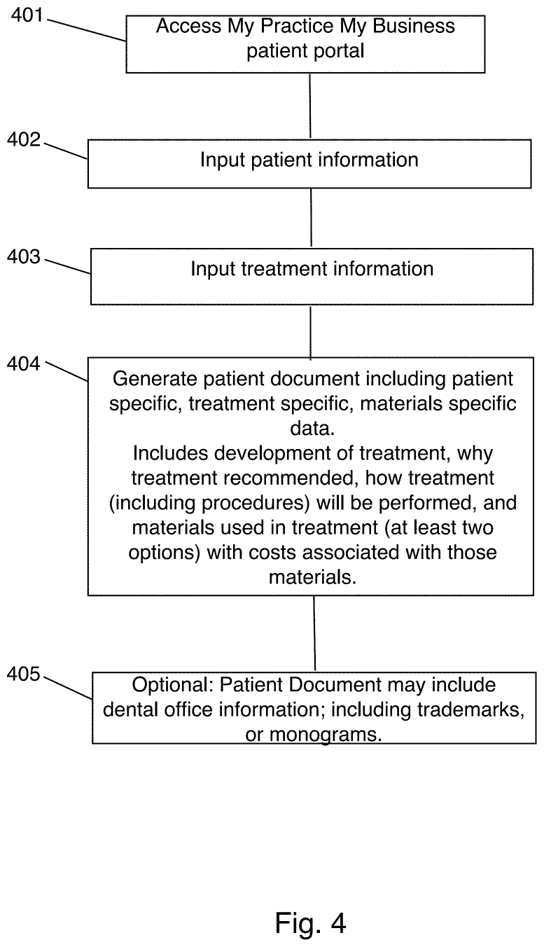

### DETAILED DESCRIPTION

This invention relates to the generation of a patient information document. The system can be used by dentists and dental offices. It will provide the patient in advance of any procedure with (a) detailed description; (b) detailed diagrams; (c) self-care information with illustrations where appropriate; and (d) answers to the most frequently asked patient questions. This data comes from a proprietary computer database carrying templates and document formats for procedure-specific patient information. The provided default data can be modified in any specific situation to provide instructions specific to a particular patient. The results lead to a condition specific, patient specific, treatment specific document.

The software associated with the system has been prepared such that:

(a) Each dental facility can tailor the procedure descriptions and diagrams as well as patient instructions to their own requirements and can save these requirements in their database template. This information can then be used as needed and, since it can be modified for a specific situation, will vastly improve upon the type of information currently distributed in pre-printed booklets and the like.

(b) Each dentist or dental practice can set up dentist-specific templates for each of the procedures he practices. These will contain personal variations on the patient instructions for the procedure and post-procedure periods along with other information such as when the sutures will be removed, and the like. This will be saved in the dentist's database under that dentist's identification number for use with patients about to undergo a particular procedure.

(c) Each multiple practice can have all of the dentist specific variations for the procedure stored in the database under each dentist's name or identification number, for use with patients that are to undergo that procedure

All of this results from the user following routines presented by the computer while the document is being prepared. This system assures the user that the resulting patient information document is complete and covers all the desired subjects.

The Patient Document

The end result of the use of this system is the preparation of a detailed patient document, which is given to the patient in advance of the procedure. This report will be in sufficient detail so that the patient is adequately educated and can make an informed decision as to the procedure to be done as well as the materials to be used. The patient will further understand the procedure to be followed and knows what is expected of him and what he can expect. This makes for a better dentist-patient relationship and tends to put the patient more at ease.

A typical individualized patient document would include:

(a) The patient condition. This describes to the patient why the dental treatment is needed)

(b) The name of the procedure to be undertaken along with a detailed description of the procedure to be undertaken. This describes in detail how the treatment will be performed.

(c) A detailed diagram or image of the procedure to be undertaken.

(d) The recommendation of the dentist. This details the material(s) and procedure(s) recommended for their condition).

(e) Information specific to their treatment (tooth number, cost, etc.).

(f) Information as to aftereffects, discomfort, healing time, and post-procedure medications.

Other or different subjects can be provided depending upon the desires of the user. Further, the subjects covered can be modified for each individual report to satisfy individual problems of a specific patient.

As can be seen, an individualized patient document such as this is valuable to the patient. It is also valuable to the dentist since he knows that all required information has been given to the patient, with nothing omitted, and the patient will have clear, understandable, written descriptions from which to make a decision as well as knowing what to expect for the procedure.

Comprehensive as these reports are, once the databases are set up, their preparation is simple and quick.

There are several types of documents that can be created from the software, including but are not limited to; Individualized Procedure documents, Consent Forms, Special Consent Forms, Post Procedure Care documents.

The following embodiments are examples and should not be construed to encompass the entirety of the invention

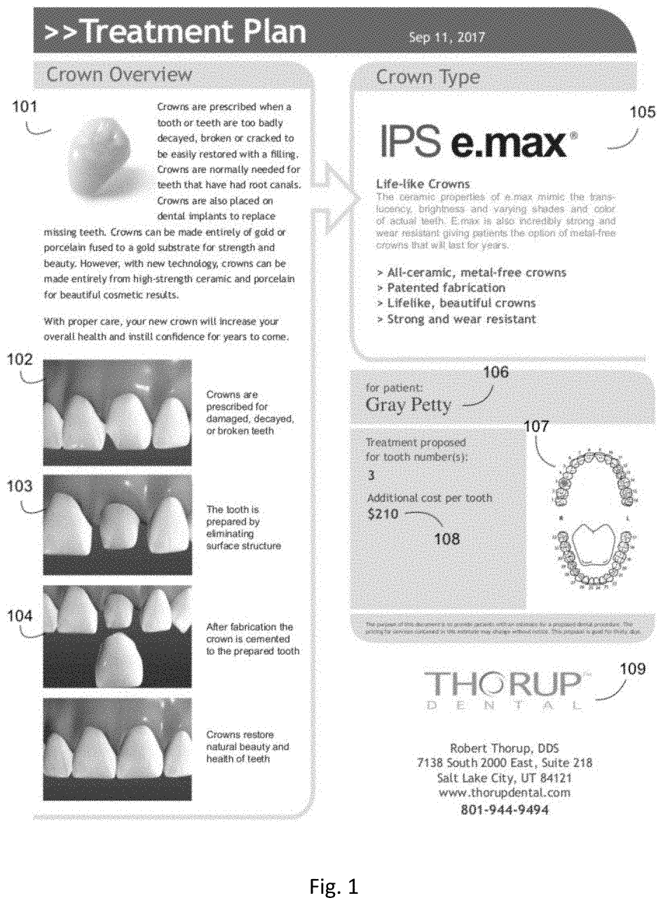

A patient comes to the dentist with a toothache. The dentist examines the tooth and determines the treatment needed. FIG. 1 depicts the treatment plan determined by the dentist. The history and general practices of the procedure are summarized at 101. The why of the procedure is described at 102. In this case, decayed teeth require fillings to restore strength and health of natural teeth. The how of the procedure is described at 103 and 104. The decayed portion of the tooth is removed from the sound tooth structure, 103. Filling material is shaped to match the look and feel of natural teeth. Traditionally, silver or amalgam material has been the filling of choice, 104. Alternative materials are described; including name of the material, and attributes of the particular material, 105. Here the filling material is Filtek, made by 3M. The attributes it claims as beneficial are; Patented nano technology, appearance of natural teeth, blends into natural tooth structure, and resists cracking and breakage, 105. The patient is named, so as to identify the patient to the procedure, 106. A diagram showing the specific teeth involved in the procedure as well as their location in the mouth allows the patient to know exactly where the procure will take place, 107. The difference in cost for the procedure including materials and labor is listed so the patient may decide which material is best for the specific situation, 108. The dentist, or dental practice may wish to include their identity, 109.

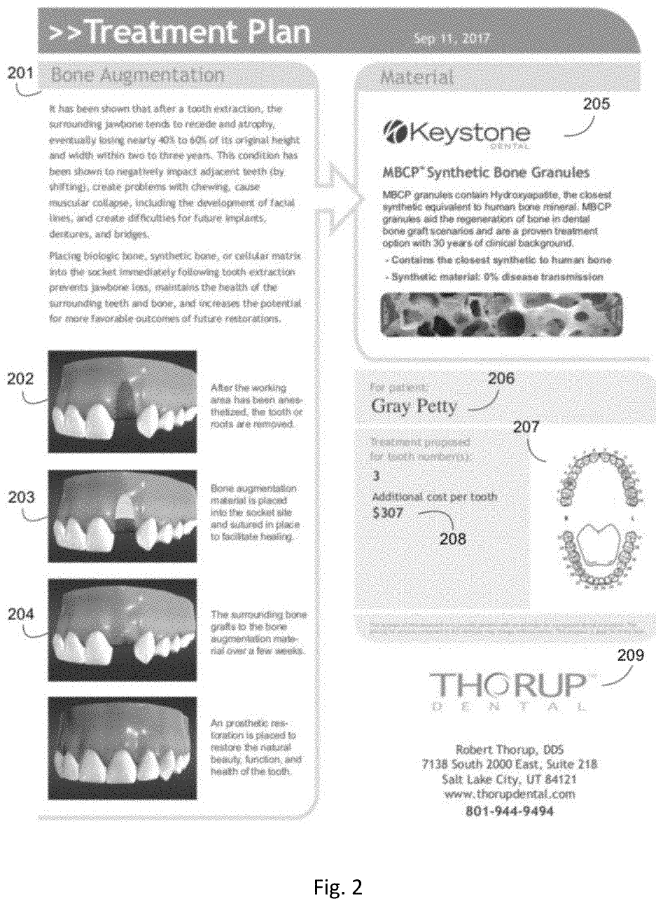

A patient comes to the dentist with a toothache. The dentist examines the tooth and determines the treatment needed. FIG. 2 depicts the treatment plan determined by the dentist. The history and general practices of the procedure are summarized at 201. The why of the procedure is described at 201. In this case, decayed teeth require removing the teeth and adding a filler to the hole. The how of the procedure is described at 203 and 204. The bone augmentation material is placed in the socket, 203. The augmentation material grafts to the bone over time, 204. Alternative materials are described; including name of the material, and attributes of the particular material, 205. Here the bone augmentation material is MBCP™ synthetic bone granules, by Keystone Dental. The attributes it claims as beneficial are; contains the closest synthetic to human bone, and synthetic material; 0% disease transmission, 205. The patient is named, so as to identify the patient to the procedure, 206. A diagram showing the specific teeth involved in the procedure as well as their location in the mouth allows the patient to know exactly where the procure will take place, 207. The difference in cost for the procedure including materials and labor is listed so the patient may decide which material is best for the specific situation, 208. The dentist, or dental practice may wish to include their identity, 209.

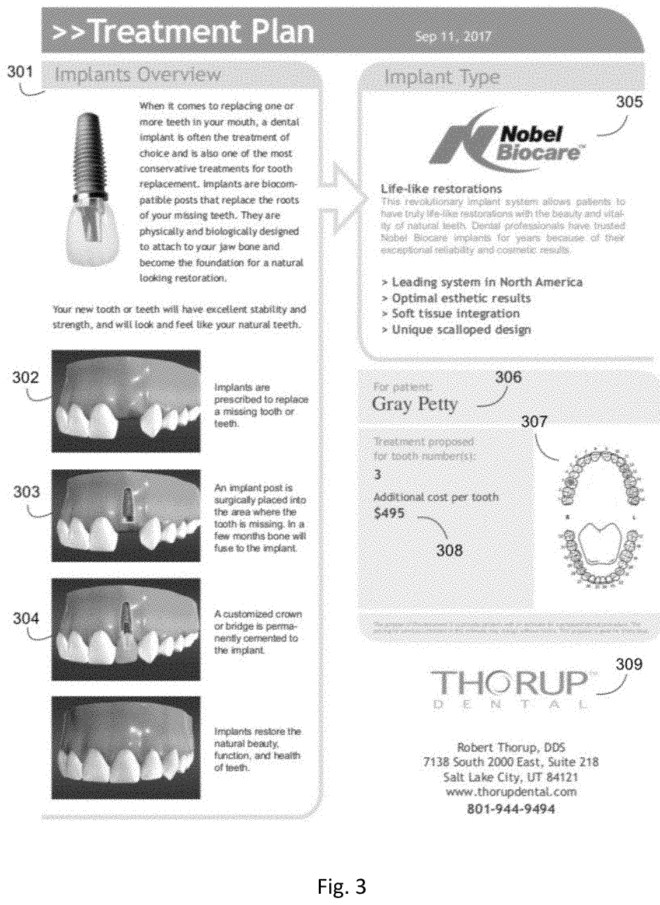

A patient comes to the dentist with a toothache. The dentist examines the tooth and determines the treatment needed. FIG. 3 depicts the treatment plan determined by the dentist. The history and general practices of the procedure are summarized at 301. The why of the procedure is described at 301. In this case, decayed teeth require removing the teeth and adding a filler to the hole. The how of the procedure is described at 303 and 304. Alternative materials are described; including name of the material, and attributes of the particular material, 305. The patient is named, so as to identify the patient to the procedure, 306. A diagram showing the specific teeth involved in the procedure as well as their location in the mouth allows the patient to know exactly where the procure will take place, 307. The difference in cost for the procedure including materials and labor is listed so the patient may decide which material is best for the specific situation, 308. The dentist, or dental practice may wish to include their identity, 309.

The dental practitioner accesses the My Practice My Business program 401. The practitioner enters the patient information, 402. The practitioner enters the treatment information, in other words identifying which treatment is to be performed, 403. The program accesses a proprietary database, which contains detailed information about each treatment, including where and when the treatment originated, why the treatment is necessary, how the treatment is to be performed, the database also contains information on the materials which can be used for the treatment, the materials include a recommended material and alternative materials, from the information input and the information in the proprietary database a patient document is generated, 404. In this way a patient specific, condition specific, and treatment specific document is generated. It is also possible for the dental practice to include their trademark or monogram on the patient document, 405, so as to identify the dental practice where the treatment was recommended.

As can be seen by the foregoing embodiments this invention results in a condition specific, patient specific and treatment specific plan. The plan may be modified by the patient according to their financial and cosmetic needs.

We claim:

1. A dental treatment plan generator comprising:
software for compiling patient details, treatment details, and condition details;
an individualized treatment analysis; and
a patient information sheet.

2. The invention of claim 1, further comprising dental materials.

3. The invention of claim 2, further comprising at least two options for the materials available for the treatment.

4. The invention of claim 3, wherein the cost difference and effectiveness of the materials is included in the information sheet.

5. The invention of claim 4, further comprising the location of the tooth in the mouth.

6. A method for generating a patient treatment plan comprising:
importing patient details into a software for compiling patient details, treatment details, and condition details;
an individualized treatment analysis; and
a patient information sheet.

7. The invention of claim 6, further comprising dental materials.

8. The invention of claim 7, further comprising at least two options for the materials available for the treatment.

9. The invention of claim 8, wherein the cost difference and effectiveness of the materials is included in the information sheet.

10. The invention of claim 9, further comprising the location of the tooth in the mouth.
